(19) United States
(12) Patent Application Publication
(10) Pub. No.: US 2005/0240275 A1
(43) Pub. Date: Oct. 27, 2005

(54) Cannulated femoral hip implant apparatus

(76) Inventor: James L. Chappuis, Marietta, GA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 11/105,881

(22) Filed: Apr. 13, 2005

Related U.S. Application Data
(60) Provisional application No. 60/561,610, filed on Apr. 13, 2004.

(51) Int. Cl. A61F 2/32; A61F 2/36

(52) U.S. Cl. 623/22.15; 623/23.26

(57) ABSTRACT

The femoral hip implant is defined by a femoral ball and a femoral stem. The femoral stem has a proximal end, a distal end and a length. The femoral stem extends from the femoral ball and terminates at the distal end. The femoral stem is arranged and configured to be received by a femur bone. A method of use is also provided.

### CROSS-REFERENCE TO RELATED APPLICATION

This application claims priority to copending U.S. provisional application entitled, “Cannulated Femoral Hip Implant Apparatus,” having Ser. No. 60/561,610, filed on Apr. 13, 2004, which is entirely incorporated herein by reference.

### FIELD OF THE INVENTION

The present invention is generally related to implants used in the medical field and more particularly, is related to a cannulated femoral hip implant apparatus.

### BACKGROUND OF THE INVENTION

Although the ability to walk, run, and freely move about is often taken for granted, an injured hip can greatly reduce a person's freedom. An injured hip can make these every day functions difficult and painful to perform.

The hip is a ball and socket joint formed at the joining of the femur and the pelvis. The femur, also known as the thighbone, terminates at a ball formed at the upper tip. The socket of the joint is formed in the pelvis. Joint function is facilitated by the ball of the femur engaging the socket of pelvis. A smooth layer of cartilage and synovial fluid, a thick lubricant, create a nearly frictionless environment in which the bones move. In a well-cushioned ball and socket joint there is no bone-on-bone contact.

A breakdown of any component of the hip results in increased pain during daily activities, such as walking or running. The most common element to breakdown is the cartilage and synovial fluid that forms the cushion between the bones. There can be many causes for such a breakdown, one of the most common being osteoarthritis in middle-aged to elderly individuals. Osteoarthritis results in dissipation of the cartilage and synovial fluid due to wear and tear over time. Other causes are rheumatoid arthritis and inflammation of the joint, which increases pressure in the joint that can damage the cartilage lining. Other causes of hip joint malfunction and injury are trauma to the joint and dislocation of the femoral ball form the pelvic socket.

Hip replacement surgery is often used to address the issue of a damaged or injured hip joint. Hip replacement surgery is now a standard procedure with a relatively high success rate. Hip replacement surgery typically entails removal of the femoral ball from the pelvic socket. The femoral ball is removed from the femur itself. A femoral canal is formed substantially axially into the remaining portion of the femur. A femoral implant, comprising a femoral stem and a femoral ball is used to replace the removed portion of the femur. The femoral stem is inserted into the femoral canal such that the femoral ball extends beyond the femur to the position where the ball of the femur extended prior to removal. The pelvic socket is reshaped in a hemisphere to substantially correspond to the dimensions of the femoral ball and is fitted with a new cup.

There are two techniques for fixing the implant parts into place, cement fixation and uncemented fixation. Cement fixation entails coating the femoral stem with bone cement prior to positioning the femoral stem in the femoral canal. With uncemented fixation, the femoral stem has a course and gritty exterior that promotes growth of the surrounding bone in the femoral canal and fixation of that bone to the femoral stem. The femoral stem can optionally be coated with a porous coating where uncemented fixation is utilized.

Fractures, known as periprosthetic fractures, to the femur below and around the femoral stem of the femoral implant, occur particularly in older osteoporatic patients. These fractures must be treated surgically. Currently, surgical treatment is a highly invasive procedure, which oftentimes requires replacement of the existing implant. Another method for securing a loosening femoral implant is use of pins, plates, etc. around the femoral stem to stabilize the implant until the fracture heals. These methods involve extensive surgery which can result in increased blood loss, infection, etc.

Thus, a heretofore unaddressed need exists in the industry to address the aforementioned deficiencies and inadequacies.

### SUMMARY OF THE INVENTION

Preferred embodiments of the present invention provide a hip implant and a method of use. Briefly described, in architecture, one embodiment of the apparatus can be implemented as follows. The femoral hip implant is defined by a femoral ball and a femoral stem. The femoral stem has a proximal end, a distal end and a length. The femoral stem extends from the femoral ball and terminates at the distal end. The femoral stem is arranged and configured to be received by a femur bone.

Preferred embodiments of the present invention can also be viewed as providing methods of use of the femoral hip implant. In this regard, one embodiment of such a method, among others, can be broadly summarized by the following steps: providing a femoral hip implant having a femoral ball, a femoral stem extending from the femoral ball and a femoral cup being arranged and configured to substantially correspond to and receive the femoral ball; providing a femoral canal in a portion of a femur bone; providing a socket in a portion of a pelvis bone; positioning the femoral cup in the socket disposed in the pelvis bone; positioning the femoral stem in the femoral canal in the femur bone; and engaging the femoral ball with the femoral cup.

Other systems, methods, features, and advantages of the present invention will be or become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

The invention can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views.

FIG. 1 illustrates a top perspective view of an embodiment of a cannulated femoral hip implant.

FIG. 2 illustrates a cut-away side view of an embodiment of a cannulated femoral hip implant illustrated in FIG. 1.

FIG. 3 illustrates a cross-sectional top view of an embodiment of a cannulated femoral hip implant illustrated in FIG. 1.

FIG. 3A illustrates a cross-sectional top view of an embodiment of a cannulated femoral hip implant illustrated in FIG. 1.

FIG. 4 illustrates a cut-away side view of an embodiment of a cannulated femoral hip implant illustrated in FIG. 1.

### DETAILED DESCRIPTION OF THE PREFERRED EMBODIMENTS

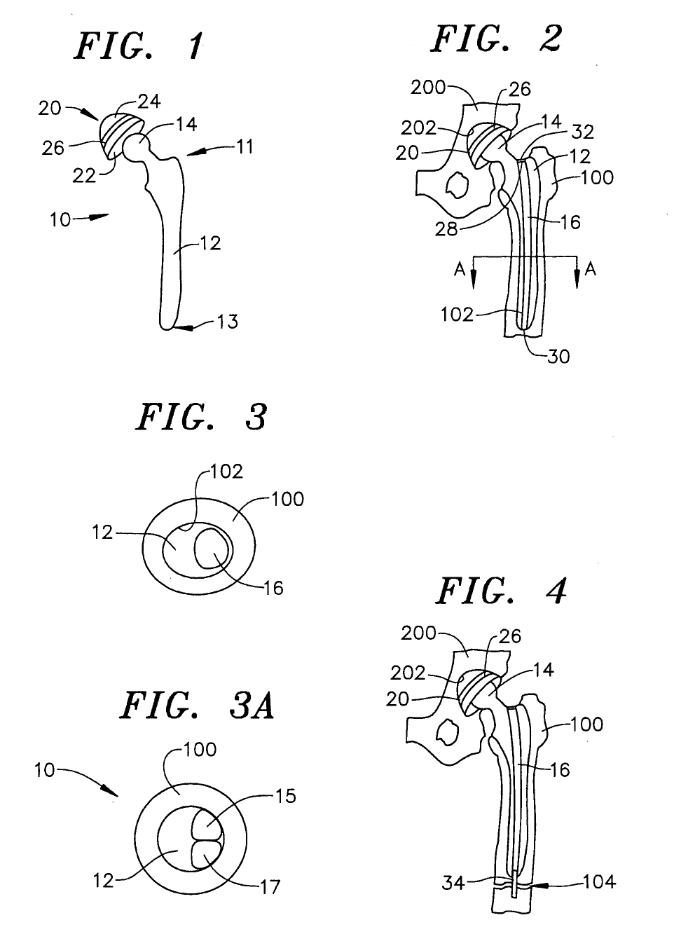

FIGS. 1 and 2 illustrate one preferred embodiment of a cannulated femoral hip implant 10. The cannulated femoral hip implant 10 comprises a femoral stem 12 and a femoral ball 14. The femoral stem 12 extends from the femoral ball 14 and is defined by a proximal end 11 and a distal end 13. In one configuration the femoral stem 12 tapers down in diameter toward the distal end 13 from the proximal end 11 such that a diameter of the femoral stem 12 toward the proximal end 11 is greater than a diameter of the femoral stem 12 toward the distal end 13. It should be understood, however, that any suitable sizing and proportioning configuration can be implemented. The femoral ball 14 has a substantially spherical shape.

As shown in FIG. 1, the cannulated femoral hip implant 10 further comprises a femoral cup 20. The femoral cup 20 is defined by an inner surface 22 and an outer surface 24. The inner surface 22 of the femoral cup 20 is concave such that the inner surface 22 substantially corresponds to the spherical shape of the femoral ball 14. It is preferable that the femoral cup 20 corresponds to the femoral ball 14 such that the femoral cup 20 can engage the femoral ball 14 while allowing free movement of the femoral ball 14 within the femoral cup 20.

The outer surface 24 of the femoral cup 20 can optionally further comprise texturing in the form of ridges 26, or the like. The textured portion of the outer surface 24 preferably engages material that surrounds the femoral cup 20, such as bone, to assist the femoral cup 20 remain in the proper position.

Referring next to FIGS. 2 and 3, a femoral stem passage 16 can be disposed in the femoral stem 12. In one configuration, the femoral stem passage 16 is disposed axially in the femoral stem 12 and extends the full length of the femoral stem 12, from the proximal end 11 to the distal end 13. In this configuration, the femoral stem passage 16 terminates at a pair of opposing openings 28 and 30. One opening 28 is disposed toward the proximal end 11 of the femoral stem and another opening 30 is disposed toward the distal end 13 of the femoral stem 12. It should be understood, however, that the femoral stem passage 16 can extend through any desired portion of the femoral stem 12.

In one configuration the femoral passage 16 is concentrically axially offset from the axial centerline of the femoral stem 12. It should be understood, however, that a concentrically axially centered femoral stem passage 16 in the femoral stem 12 is also within the spirit of the present invention. The femoral stem passage 16 can accommodate various means for fixing the femoral stem 12 into position in a femur bone 100. As example, without limitation, the femoral stem passage 16 can accommodate a BMP-style gel, pins, or the like.

The cannulated femoral hip implant 10 can further comprise a passage cap 32. The passage cap 32 is arranged and configured to releasably engage the opening 28 of the femoral stem passage 16 disposed toward the proximal end 11 of the femoral stem 12. In one configuration the passage cap 32 can be press-fit to engage the opening 28; however, it should be understood that any manner that allows for releasable engagement of the passage cap 32 with the opening 28 is acceptable.

Another embodiment of the cannulated femoral hip implant 10 is illustrated in FIG. 3A. A pair of femoral stem passages 15 and 17 are disposed within the femoral stem 12. Each of the pair of femoral stem passages 15 and 17 can be used as guide channels for fixation of a pin in use, and as discussed in further detail hereinbelow. The pair of femoral stem passages 15 and 17 are axially offset from the axial centerline of the femoral stem 12. It should be understood, however, that the pair of femoral stem passages 15 and 17 can be disposed in any desired portion of the femoral stem 12. Although the cannulated femoral hip implant 10 is disclosed herein having a single femoral stem passage 16 in one embodiment and a pair of femoral stem passages 15 and 17 in another embodiment, it should be understood that the cannulated femoral hip implant 10 can comprise any suitable number of femoral stem passages disposed in the femoral stem 12.

In one method of use of the cannulated femoral hip implant 10, a socket 202 is provided in a portion of a pelvic bone 200 that is substantially adjacent the femur bone 100 in which the femoral canal 102 is provided. It is preferable that the socket 202 substantially corresponds to the femoral cup 20 of the cannulated femoral hip implant 10. The femoral cup 20 is positioned in the socket 202 such that the outer surface 24 of the femoral cup 20 is in contact with the portion of the pelvic bone 200 that forms the socket 202.

Optional ridges 26 disposed on or formed into the outer surface 24 of the femoral cup 20 are positioned in contact with a portion of the pelvic bone 200 that forms the socket 202. The ridges 26 act to deter disengagement of the femoral cup 20 from the socket 202. It should be understood that, although ridges 26 are disclosed herein, any form of texturing is within the scope of the present invention.

A femoral canal 102 is provided in a portion of a femur bone 100. It is preferable that the femoral canal 102 substantially corresponds to the femoral stem 12 of the implant 10. The femoral stem 12 is positioned into the femoral canal 102 such that the femoral stem 12 extends axially along a length of the femoral canal 102.

Any suitable means of fixing the femoral stem 12 to the femur bone 100 can be inserted into the femoral stem passage 16, such as BMP-type gel, pins, or the like. A passage cap 32 can be fixed into position over opening 28.

In another method of use, the cannulated femoral hip implant can be used to repair a femur fracture 104. As illustrated in FIG. 4, a fixing means, such as a pin 34, can be inserted into the femur stem passage 16 such that pin 34 extends from a portion of the femur bone surrounding the femur canal 12 to a portion of the femur 100 below a femur fracture 104. The pin 34 is engaged into position after the femoral stem 12 and the femoral cup 20 are positioned as desired and as described above.

It should be emphasized that the above-described embodiments of the present invention, particularly, any “preferred” embodiments, are merely possible examples of implementations, merely set froth for a clear understanding of the principles of the invention. Many variations and modifications may be made to the above-described embodiment(s) of the invention without departing substantially from the spirit and principles of the invention. All such modifications and variations are intended to be included herein within the scope of this disclosure and the present invention and protected by the following claims.

1. A femoral hip implant comprising:
a femoral ball; and
a femoral stem having a proximal end, a distal end and a length; said femoral stem extending from said femoral ball and terminating at said distal end;
wherein said femoral stem is arranged and configured to be received by a femur bone.

2. A femoral hip implant of claim 1, further comprising:
a femoral passage disposed axially in said femoral stem;
wherein said femoral passage comprises and opening in said femoral stem.

3. A femoral hip implant of claim 2, wherein said femoral passage extends said length of said femoral stem to said distal end of said femoral stem.

4. A femoral hip implant of claim 2, further comprising:
a passage cap being arranged and configured to removably engage said opening of said femoral passage in said femoral stem.

5. A femoral hip implant of claim 1, further comprising:
a femoral cup being arranged and configured to substantially correspond to said femoral ball;
wherein said femoral cup is arranged and configured to be received by a portion of a pelvis bone.

6. A femoral hip implant of claim 5, wherein said femoral cup is arranged and configured to substantially correspond to said femoral ball such that said femoral ball substantially freely moves within said femoral cup.

7. A femoral hip implant, comprising:
a femoral cup being arranged and configured to be engaged by a portion of a pelvis bone;
a femoral ball being arranged and configured to substantially correspond to said femoral cap;
a femoral stem extending from said femoral ball said femoral stem being arranged and configured to engage a portion of a femur bone; and
a femoral passage disposed axially in said femoral stem;
wherein said femoral passage comprises and opening in said femoral stem.

8. A femoral hip implant of claim 7, wherein said femoral stem is fixed to a portion of a femur bone via said femoral passage.

9. A femoral hip implant of claim 7, further comprising:
a passage cap being arranged and configured to removably engage said opening of said femoral passage.

10. A method of using a femoral hip implant, comprising the steps of:
providing a femoral hip implant having:
a femoral ball;
a femoral stem extending from said femoral ball; and
a femoral cup being arranged and configured to substantially correspond to and receive said femoral ball;
providing a femoral canal in a portion of a femur bone;
providing a socket in a portion of a pelvis bone;
positioning said femoral cup in said socket disposed in the pelvis bone;
positioning said femoral stem in said femoral canal in the femur bone; and
engaging said femoral ball with said femoral cup.

11. The method of claim 10, further comprising the steps of:
providing a femoral stem passage having an opening in said femoral stem; and
fixing said femoral stem to the portion of the femur bone.

12. The method of claim 11, further comprising the steps of:
providing a passage cap being arranged and configured to releasably engage said opening of femoral stem passage.

13. A method of using a femoral hip implant, comprising the steps of:
providing a femoral hip implant having:
a femoral ball; and
a femoral stem extending from said femoral ball;
providing a femoral canal in a portion of said femur bone; and
positioning said femoral stem in said femoral canal.

14. The method of claim 13, further comprising the step of:
providing a femoral passage extending axially through said femoral stem;
wherein said femoral passage comprises an opening in said femoral stem.

15. The method of claim 13, further comprising the step of:
fixing said femoral stem to said femoral canal through said femoral passage.

16. The method of claim 13, further comprising the step of:
engaging said femoral ball with a portion of a pelvis bone.

17. The method of claim 16, wherein the step of engaging further comprises the steps of:
providing a socket in a portion of the pelvis bone;
providing a femoral cup, wherein said femoral cup is arranged and configured to substantially correspond to and engage said femoral ball;
positioning said femoral cup in the socket; and
engaging said femoral cup with said femoral ball.
